(19) United States
(12) Patent Application Publication
(10) Pub. No.: US 2014/0154269 A1
(43) Pub. Date: Jun. 5, 2014

(54) TARGETED NANOVECTORS AND THEIR USE FOR TREATMENT OF BRAIN TUMORS

(71) Applicants: James M. Tour, Bellaire, TX (US); Jacob Berlin, Monrovia, CA (US); Daniela Marcano, Houston, TX (US); David S. Baskin, Houston, TX (US); Martyn A. Sharpe, Houston, TX (US)

(72) Inventors: James M. Tour, Bellaire, TX (US); Jacob Berlin, Monrovia, CA (US); Daniela Marcano, Houston, TX (US); David S. Baskin, Houston, TX (US); Martyn A. Sharpe, Houston, TX (US)

(73) Assignees: The Methodist Hospital Research Institute, Houston, TX (US); William Marsh Rice University, Houston, TX (US)

(21) Appl. No.: 14/114,051

(22) PCT Filed: Apr. 26, 2012
(86) PCT No.: PCT/US12/35267

§ 371 Date: Jan. 13, 2014

Related U.S. Application Data
(60) Provisional application No. 61/479,220, filed on Apr. 26, 2011.

(51) Int. Cl. A61K 9/00

(52) U.S. Cl. CPC A61K 9/0085
USPC 424/174.1; 514/44 A; 514/44 R; 424/649; 514/19.3

(57) ABSTRACT

In some embodiments, the invention pertains to therapeutic compositions for treating a brain tumor. Such therapeutic compositions generally comprise: (1) a nanovector; (2) an active agent associated with the nanovector with activity against brain tumor cells; and (3) a targeting agent associated with the nanovector with recognition activity for a marker of the brain tumor cells. In some embodiments, the active agent and the targeting agent are non-covalently associated with the nanovector. Additional embodiments of the present invention pertain to methods of treating a brain tumor in a subject (e.g., a human being) by administering the aforementioned therapeutic compositions to the subject. Further embodiments of the present disclosure pertain to methods of formulating therapeutic compositions for treating a brain tumor in a subject in a personalized manner.

### CROSS-REFERENCE TO RELATED APPLICATIONS

This application claims priority to U.S. Provisional Patent Application No. 61/479,220, filed on Apr. 26, 2011. This application is also a continuation-in-part of Patent Cooperation Treaty Application No. PCT/US2010/054321, filed on Oct. 27, 2010, which claims priority to U.S. Provisional Application No. 61/255,309, filed on Oct. 27, 2009. The entirety of each of the aforementioned applications is incorporated herein by reference.

### STATEMENT REGARDING FEDERALLY SPONSORED RESEARCH

This invention was made with government support under U.S. Army Grant No. W81XWH-08-2-0143, awarded by the U.S. Department of Defense; and NSF Grant No. EEC-0647452, awarded by the National Science Foundation. The Government has certain rights in the invention.

### BACKGROUND

Current methods to treat brain tumors suffer from various limitations. Such limitations include an inability to effectively and specifically deliver desired drugs to tumor sites. Such limitations are further escalated when desired drugs are hydrophobic, and when the tumor displays resistance to multiple drugs. Additional obstacles include lack of effective methods of making personalized drug delivery compositions that effectively target a desired brain tumor in a particular subject. Therefore, more efficient and effective approaches to targeted drug delivery are desired for treating various brain tumors.

### BRIEF SUMMARY

In some embodiments, the present disclosure pertains to therapeutic compositions for treating a brain tumor. Such therapeutic compositions generally comprise: (1) a nanovector; (2) an active agent associated with the nanovector that has activity against brain tumor cells; and (3) a targeting agent associated with the nanovector with recognition activity for a marker of the brain tumor cells. In some embodiments, the active agent and the targeting agent are non-covalently associated with the nanovector. In some embodiments, one or more of such components are covalently associated with the nanovector.

In some embodiments, the nanovector includes at least one of single-walled nanotubes, double-walled nanotubes, triple-walled nanotubes, multi-walled nanotubes, ultra-short nanotubes, graphene, graphene nanoribbons, graphite, graphite oxide nanoribbons, carbon black, hydrophilic carbon cluster (HCC), and combinations thereof. In some embodiments, the nanovector includes hydrophobic domains and hydrophilic domains. In more specific embodiments, a hydrophobic active agent is associated with the hydrophobic domain.

In some embodiments, the nanovector is functionalized with a plurality of solubilizing groups, such as polyethylene glycols, poly(p-phenylene oxide), polyethylene imines, and combinations thereof. In more specific embodiments, the nanovector is an ultra-short single-walled nanotube that is functionalized with a plurality of solubilizing groups, such as a poly(ethylene glycolated) hydrophilic carbon cluster (PEG-HCC).

In some embodiments, the active agent is a hydrophobic compound. In some embodiments, the active agent includes at least one of small molecules, proteins, DNA, antisense oligonucleotides, miRNA, siRNA, aptamers, and combinations thereof. In more specific embodiments, the active agent includes at least one of Cis-platin, Paclitaxel, SN-38, Vinblastine, Daunorubicin, Docetaxel, Iadarubicin, Oxaliplatin, 1,2,3,4-tetrahydronaphthalene-2,3-diamine, 2,2-dichloro-octahydrocyclohexa 1,3-diaza-2-platinacyclopentane, 2,2-dichloro-hexahydronaphtho 1,3-diaza-2-platinacyclopentane, 4,4-dichloro-3,5-diaza-4platinatetracycloheptadecahexaene, nitrogen mustards, spermine mustards, estrogen mustards, cholesterol mustards, and combinations or derivatives thereof.

In some embodiments, the targeting agent includes at least one of antibodies, proteins, RNA, DNA, aptamers, small molecules, dendrimers, and combinations thereof. In more specific embodiments, the targeting agent is an antibody directed against a marker of the brain tumor cells.

In some embodiments, the marker of the brain tumor cells is an epitope on a surface of the brain tumor cells, such as glial fibrillary acidic protein (GFAP). In some embodiments, the marker is a receptor on a surface of the brain tumor cells, such as epidermal growth factor receptors, cytokine receptors, interleukin receptors, and combinations thereof.

Additional embodiments of the present disclosure pertain to methods of treating a brain tumor in a subject (e.g., a human being) by administering the aforementioned therapeutic compositions to the subject. Further embodiments of the present disclosure pertain to methods of formulating therapeutic compositions for treating a brain tumor in a subject by: (1) isolating brain tumor cells from the subject; (2) determining expression levels of one or more markers of the brain tumor cells; and (3) formulating one or more therapeutic compositions that include (a) a nanovector; (b) an active agent associated with the nanovector; and (c) a targeting agent associated with the nanovector with recognition activity for a marker of the brain tumor cells. In some embodiments, the targeting agent is selected based on the determined expression levels of the one or more markers of the brain tumor cells. Further embodiments of such methods may also include a step of determining the susceptibility of the brain tumor cells to one or more active agents and selecting an active agent in the therapeutic composition based on the determined susceptibility. In some cases, this approach of treatment can be termed “personalized medicine.”

The methods and compositions of the present disclosure can be used to treat various brain tumors in a specific, personalized and effective manner. In some embodiments, the treated brain tumor may include, without limitation, gliomas, glioblastomas, astrocytomas, neuroblastomas, retinoblastomas, meduloblastomas, oligodendrogliomas, ependymomas, choroid plexus papillomas, meningiomas, pituitary adenomas, and combinations thereof. In more specific embodiments, the brain tumor to be treated is a primary glioblastoma multiforme (GBM).

### BRIEF DESCRIPTION OF THE FIGURES

FIG. 1 shows active agents that could be used to treat brain tumors in accordance with various embodiments of the present disclosure. The active agents are listed in FIGS. 1A and 1B in the order of increasing hydrophobicity. The structures of additional active agents are illustrated in FIG. 1C.

FIG. 2 illustrates a scheme for formulating an individualized therapeutic composition for treating brain tumors.

FIG. 3 illustrates the epitope mapping of glioblastoma multiforme (GBM) cultures. Three control cultures of GBM were stained with Hoechst prior to fixation in paraformaldehyde (PFA). The treated cells were then incubated with monoclonal antibodies to glial fibrillary acidic protein (GFAP) (FIG. 3A), interleukin-13 Receptor (IL-13R) (FIG. 3B), and epidermal growth factor receptor (EGFR) (FIG. 3C). Next, the cells were treated with a red fluorescently labeled anti-mouse antibody. FIG. 3D shows the binding of a therapeutic composition to these cells. The therapeutic composition consisted of a polyethylene glycol (PEG) functionalized hydrophilic carbon cluster (HCC) that was non-covalently associated with anti-GFAP antibodies and SN-38 (GFAP_{AB}/SN-38/PEG-HCC). This panel shows that the anti-GFAP antibodies on the PEG-HCCs were co-localized with the GBM cell surface after a 1 hour incubation, as in FIG. 3A. All panels are images at 20× magnification.

FIG. 4 shows data indicating that GFAP_{AB}/SN-38/PEG-HCCs kill GBM primary cultures. FIG. 4A shows that three cell viability measurements indicate the killing of GBMs by GFAP_{AB}/SN-38/PEG-HCCs. The tests included ddTUNEL (white bars), Dead Green staining (gray bars) and Hoechst staining (striped bars). FIG. 4B indicates that, based on average levels of living GBM cells (left), from ddTUNEL, Dead Green, and Hoechst staining, show that the individual therapeutic composition components, PEG-HCCs, GFAPAB/PEG-HCCs, and SN-38/PEG-HCCs are non-toxic, whereas the combined treatment, in the form of GFAP_{AB}/SN-38/PEG-HCCs, causes significant cell death. Additionally, changes in cell protein mass, using the BCA method (right panel), correlate with viable cell numbers determined using viability stains in fixed cells, using the lethal uncoupling agent carbonyl cyanide chlorophenyl hydrazone (CCCP) to establish the minimum cellular protein levels. FIG. 4C is a comparison of SN-38 toxicity when presented to GBM in solution or as GFAP_{AB}/SN-38/PEG-HCCs. SN-38 is insoluble in water, so it had to be delivered in ethanol and was compared to an ethanol control. Thus, changes in protein mass after 24 h treatment with SN-38/PEG-HCCs (white bars) and SN-38 (black bars) were compared to saline or ethanol only controls, respectively. SN-38/PEG-HCCs are not toxic up to 20 μM SN-38, whereas aqueous SN-38 has an LD₅₀ of ˜8 μM. In all figures, n=8 wells, and the error bars are equal to the SD.

FIG. 5 shows data indicating that PEG-HCCs are not toxic towards confluent cultures of human cortical neurons (HCN), normal human astroctyes (NHA) and GBM following 24 h exposure to high concentrations of PEG-HCC, as shown by cell protein levels (n=8 wells; error bars SD).

FIG. 6 shows the versatility of therapeutic compositions (e.g., GFAP_{AB}/SN-38/PEG-HCCs) in having broad antibody/active agent specificity and lethality towards a range of GBMs.

FIG. 6A shows the dose response curve of three different GBMs (dashed lines) and one anaplastic astrocytoma (solid line) toward GFAP_{AB}/SN-38/PEG-HCCs, measured at 24 h. FIG. 6B shows treatment with therapeutic compositions using three hydrophobic active agents: Vin (□), Doc (o) and SN-38 (⋄). The active agents were presented to GBMs for 24 h within PEG-HCCs, and targeted to the tumor antigen, EGFR, by an IgG. FIG. 6C shows that astrocytes are insensitive to therapeutic compositions and their individual components, as shown by protein measurement following 24 h incubation. The white bar on left represents the control experiment. Incubation of NHA with EGFR_{AB} and EGFR_{AB}/PEG-HCCs (next two black bars) and then with EGFR_{AB} in the absence (gray bars) and presence (black bars) of PEG-HCCs ±active agent (5 μM) causes no change in protein mass.

FIG. 7 shows the effects of Vin, Doc and SN-38 on GBMs when they were incorporated into therapeutic compositions individually or in combination. The results were measured using six different death markers. All active agents were at a final concentration of 0.5 μM. The upper row shows 3′OH DNA ends, Dead Green and Hoechst DNA staining. The middle row shows mitochondrial membrane potential. The bottom row shows blunt ended, lethal, DNA breaks and Caspase-3 activity. All of the figures are at 20× magnification. The side bars show the calibration scale for each fluorophore.

FIG. 8 shows the pattern of cell death in three GBMs (FIGS. 8A-8C) and one anaplastic astrocytoma cell culture (FIG. 8D) that were treated with the therapeutic compositions in FIG. 7. All figures are at 20× magnification. However, the bottom row is expanded further by 4× and uses a non-linear scale (G=0.5). In the bottom panel, DNA staining is shown by Hoechst at higher magnification (10 μm scale bar), which allows easier identification of the known outcomes of the microtubule disrupting active agents (Doc and Vin). Mitotic catastrophe is found using both active agents, with many nuclei having atypical morphology. The distorted/cog wheel shaped nuclei, indicative of cell cycle arrest at the G2/M phase, are visible in the Doc treated cancer cells.

FIG. 9 provides results indicating that therapeutic compositions (including ones with three active agents) are not overly toxic towards astrocytes (FIG. 9B) and neurons (FIG. 9C), but are highly toxic towards GBMs (FIG. 9A). The living and dead cell numbers resulting from treatment using 0.5 μM of active agents (Vin, Doc and SN-38) targeted with monoclonal antibodies to GBM surface antigens (IL-13R, EGFR and GFAP) are shown. Black bars are % control living cells, white bars are % dead cells. (GBM: n=8 wells; NHA: n=8 wells; HCN: n=4 wells; error bars SD in all cases).

FIG. 10 summarizes the effects of 24 h treatments of therapeutic composition and trident therapy treatments in human cortical neurons (HCN), as measured using the BCA protein method. On the left are four HCN controls, 100 μM carbonyl cyanide chlorophenyl hydrazone (CCCP) (100% cell death), saline vehicle, PEG-HCC and PEG-HCC bound to monoclonal antibodies toward GFAP, IL-13R or EGFR PEG-HCC. Treatments with three different therapeutic compositions are also shown, where PEG-HCCs were loaded with the following active agents: Vin, Doc or SN-38, either with antibodies (gray) or without antibodies (black). The final pairing shows that trident therapy without antibodies (black) is no more toxic than when active agent/PEG-HCC is presented to cells with all three antibodies. Protein levels were measured in 7 wells ±SD. Only in the CCCP positive control and the untargeted/targeted trident therapy were the HCN protein levels less than the control levels (p<0.05, ANOVA and Turkey post-hoc test).

FIG. 11 shows that SN-38/PEG-HCCs are not toxic towards GBMs following 24 hour exposure to very high concentrations of PEG-HCCs, but that SN-38 is toxic with an LD₅₀ of 5 to 10 μM. In FIG. 11A, the effects of SN-38 delivered as an ethanolic solution are compared with the same concentration delivered as SN-38/PEG-HCC. Hoechst viability staining was used to measure the number of live and dead cells following 24 h incubation with SN-38 or SN-38/PEG-HCC. FIG. 11B shows the dose dependency of two primary GBM cultures treated with ethanolic SN-38, indicating that the LD₅₀ is approximately 8 μM (n=8 wells; error bars SD).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Antibodies and proteins have been used to target the delivery of anti-cancer drugs. However, several difficulties have presented themselves for the development of effective targeted anti-cancer therapies. For instance, direct covalent-bond attachment of an active agent to a targeting agent (such as an antibody) often requires a significant synthetic effort. Perhaps more significantly, it can be challenging to attach a sufficient amount of the active agent to each targeting agent without compromising the solubility or activity of the targeting agent (e.g., antibody). An alternative strategy is to make use of a third body platform, such as a dendrimer, to increase the loading of active agent relative to the targeting agent. This approach entails a much more difficult synthetic effort, as both the active agent and the targeting agent can be attached to the platform.

Additional limitations with current cancer therapies include an inability to effectively and specifically deliver desired drugs to tumor sites. Such limitations are further escalated when desired drugs are hydrophobic, and when the tumor displays resistance to multiple drugs. Additional obstacles include lack of effective methods of making personalized drug delivery compositions that effectively target a desired brain tumor in a particular subject. Therefore, more efficient and effective approaches to targeted drug delivery are desired for treating various brain tumors. The present disclosure addresses these needs.

In some embodiments, the present disclosure provides therapeutic compositions for treating a brain tumor. In some embodiments, the therapeutic compositions comprise at least: (1) a nanovector; (2) an active agent associated with the nanovector that has activity against brain tumor cells; and (3) a targeting agent associated with the nanovector that has recognition activity for a marker of the brain tumor cells. Further embodiments of the present disclosure pertain to methods of making the above-mentioned therapeutic compositions and using them to treat brain tumors in subjects, such as patients.

Reference will now be made to more specific and non-limiting embodiments of the present disclosure. Additional support for the embodiments of the present disclosure can also be found in the following of Applicants' patent applications: PCT/US2008/078776, entitled “Water Soluble Carbon Nanotube Compositions for Drug Delivery and Medical Applications”; and PCT/US2010/054321, entitled “Therapeutic Compositions and Methods for Targeted Delivery of Active Agents.” Also see U.S. patent application Ser. Nos. 12/245,438 and 12/280,523.

Therapeutic Compositions

Various embodiments of the present disclosure pertain to therapeutic compositions for treating one or more brain tumors. In some embodiments, the therapeutic compositions of the present disclosure generally comprise: (1) a nanovector; (2) an active agent associated with the nanovector, where the active agent has activity against brain tumor cells; and (3) a targeting agent associated with the nanovector, where the targeting agent has recognition activity for a marker of the brain tumor cells. As set forth in more detail below, such therapeutic compositions can have various embodiments and arrangements. For instance, various nanovectors, active agents and targeting agents may be utilized. Furthermore, the therapeutic compositions of the present disclosure may have multiple active agents.

Nanovectors

Nanovectors suitable for use in the therapeutic compositions of the present disclosure generally refer to particles that are capable of associating with an active agent and a targeting agent. Nanovectors in the present disclosure also refer to particles that are capable of delivering an active agent to a targeted area.

In some embodiments, suitable nanovectors include, without limitation, single-walled nanotubes (SWNTs), double-walled nanotubes (DWNTs), triple-walled nanotubes (TWNTs), multi-walled nanotubes (MWNTs), ultra-short nanotubes, ultra-short single-walled nanotubes (US-SWNTs), hydrophilic carbon clusters (HCCs), graphene nanoribbons, graphite, graphite oxide nanoribbons, carbon black, derivatives thereof, and combinations thereof.

In some embodiments, the nanovectors of the present disclosure may be modified in various ways. For instance, in some embodiments, the nanovectors of the present disclosure may be oxidized. In some embodiments, the nanovectors of the present disclosure may be functionalized with one or more molecules, polymers, chemical moieties, solubilizing groups, functional groups, and combinations thereof. For instance, in some embodiments, the nanovectors of the present disclosure may be functionalized with ketones, alcohols, epoxides, carboxylic acids, and combinations thereof.

In more specific embodiments, the nanovectors of the present disclosure may be functionalized with a plurality of solubilizing groups. In further embodiments, the solubilizing groups may include at least one of polyethylene glycols (PEGs), polypropylene glycol (PPG), poly(p-phenylene oxide) (PPOs), polyethylene imines (PEI), poly(vinyl alcohol) (PVA), poly(acrylic acid) (PAA), poly(vinyl amine) and combinations thereof. In more specific embodiments, the nanovectors of the present disclosure can include PEG-functionalized HCCs (i.e., PEG-HCCs, as described in more detail below).

The nanovectors of the present disclosure may also have various properties. For instance, in some embodiments, the nanovector may be hydrophilic (i.e., water soluble). In some embodiments, the nanovectors of the present disclosure may have both hydrophilic portions and hydrophobic portions. For instance, in some embodiments, the nanovectors of the present disclosure may have a hydrophilic domain (e.g, a hydrophilic surface) and a hydrophobic domain (e.g., a hydrophobic cavity). The nanovectors of the present disclosure can also be engineered to possess both hydrophobic and hydrophilic domains, combining high aqueous solubility with the ability to adsorb hydrophobic compounds. In some embodiments, this duality of hydrophilic and hydrophobic domains can result in the formation of structures resembling micelles or liposomes. Such structures can in turn further entrap active agents for delivery to a desired site.

In more specific embodiments, the nanovectors of the present disclosure include US-SWNTs. US-SWNTs are also referred to as hydrophilic carbon cluster (HCCs). Therefore, for the purposes of the present disclosure, US-SWNTs are synonymous with HCCs. In some embodiments, HCCs can include oxidized carbon nanoparticles that are about 30 nm to about 40 nm long, and approximately 1-2 nm wide.

In some embodiments, US-SWNTs (i.e., HCCs) may be produced by reacting SWNTs in fuming sulfuric acid with nitric acid to produce a shortened carbon nanotube characterized by opening of the nanotube ends. Such methods are disclosed in Applicants' co-pending U.S. patent application Ser. No. 12/280,523, entitled “Short Functionalized, Soluble Carbon Nanotubes, Methods of Making Same, and Polymer Composites Made Therefrom.” This may be followed by the functionalization of the plurality of carboxylic acid groups. In some embodiments, the HCC may be an oxidized graphene.

In some embodiments, the HCCs may be functionalized with one or more solubilizing groups, such as PEGs, PPGs, PPOs, PEIs, PVAs, PAAs, poly(vinyl amines) and combinations thereof (as previously described). In more specific embodiments, the nanovectors of the present disclosure may include polyethylene glycol-functionalized HCCs (PEG-HCCs). Various PEG-HCCs and methods of making them are disclosed in the following articles and applications:

Berlin et al., ACS Nano 2010, 4, 4621-4636; Lucente-Schultz et al., J. Am. Chem. Soc. 2009, 131, 3934-3941; Chen et al., J. Am. Chem. Soc. 2006, 128, 10568-10571; Stephenson, et al., Chem. Mater. 2007, 19, 3491-3498; Price et al., Chem. Mater. 2009, 21, 3917-3923; PCT/US2008/078776; and PCT/US2010/054321.

In various embodiments, PEG-HCCs (and other functionalized forms of HCCs) may have various advantageous properties for use as nanovectors. For instance, PEG-HCCs (and other functionalized forms of HCCs) may demonstrate low biological toxicity with clearance mainly through the kidneys. PEG-HCCs (and other functionalized forms of HCCs) may also contain hydrophobic domains that can be non-covalently loaded with active agents, such as hydrophobic active agents. In addition, PEG-HCCs (and other functionalized forms of HCCs) can have an ability to strongly bind to various targeting agents (such as monoclonal or IgG-type antibodies) without significantly interfering with the activity of the targeting agents. Thus, active agent-loaded PEG-HCCs (and other functionalized forms of HCCs) combined with a targeting agent can be used to bind to a chosen cell surface antigen and deliver a hydrophobic, lipophilic active agent into or on cells that express a selected epitope.

Other suitable PEGylated or functionalized carbon nanomaterials can also be used as nanovectors. Non-limiting examples include PEGylated graphite oxide nanoribbons (PEG-GONR), PEGylated oxidized carbon black (PEG-OCB), and PEGylated carbon black (PEG-CB). Additional suitable nanovectors, including PEG-HCCs, are disclosed in U.S. patent application Ser. No. 12/245,438; PCT/US2008/078776; and PCT/US2010/054321. The use of other suitable nanovectors not disclosed here can also be envisioned.

Active Agents

Active agents of the present disclosure generally refer to biologically active compounds, such as compounds that have activity against brain tumor cells (e.g., anti-apoptoic activity, anti-proliferative activity, anti-oxidative activity, etc.). For instance, in various embodiments, active agents of the present disclosure may refer to anti-cancer drugs, chemotherapeutics, antioxidants, and anti-inflammatory drugs. Furthermore, the active agents of the present disclosure may be derived from various compounds. For instance, in various embodiments, the active agents of the present disclosure can include, without limitation, small molecules, proteins, aptamers, DNA, anti-sense oligo nucleotides, miRNA, siRNA, and combinations thereof.

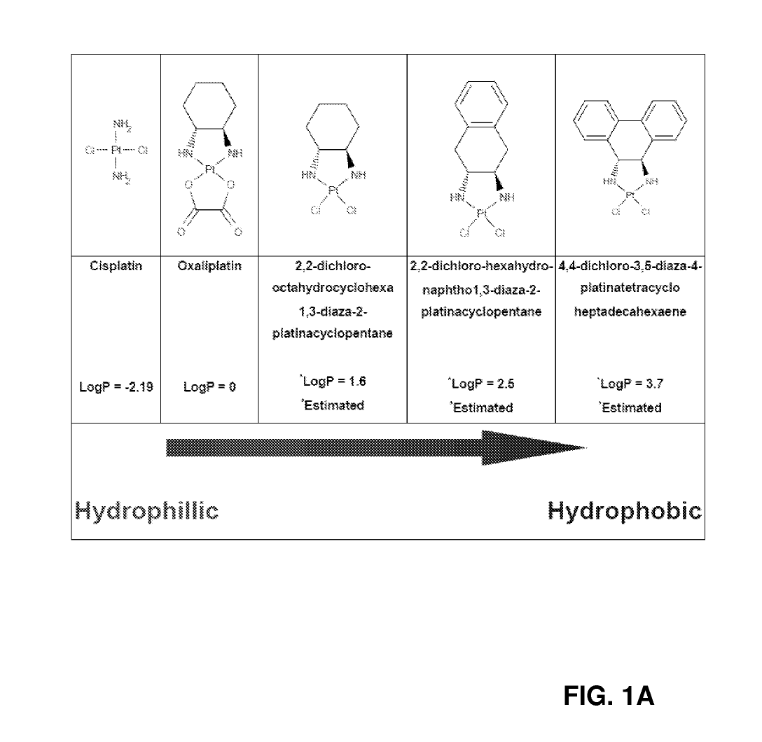

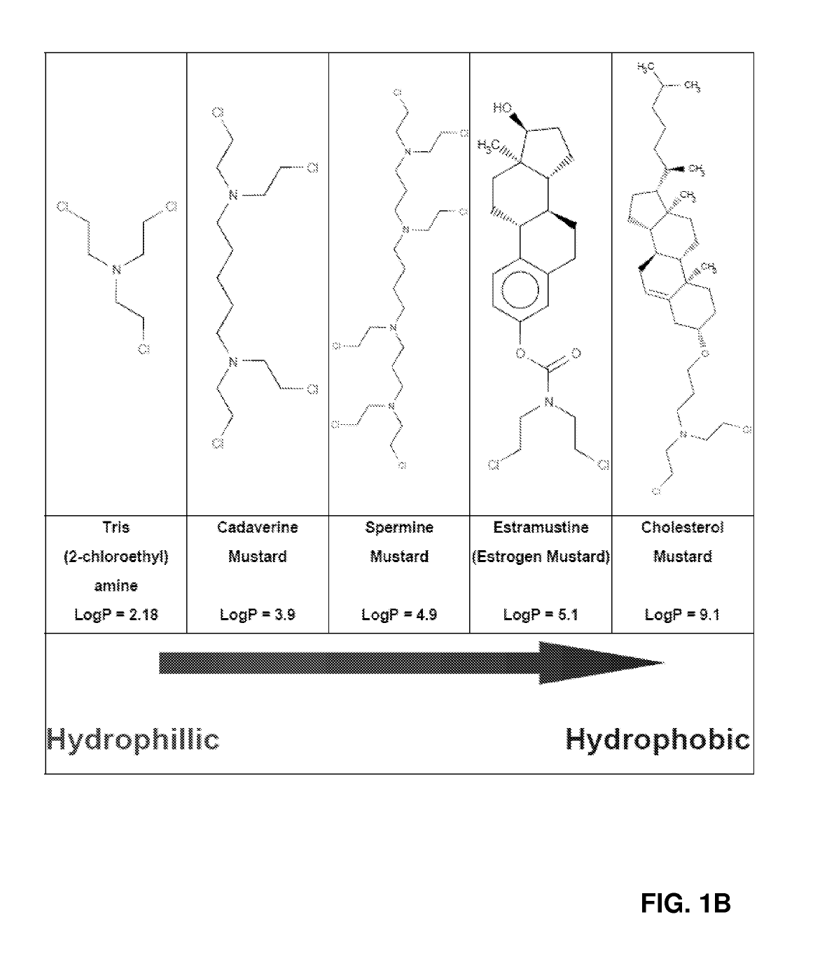

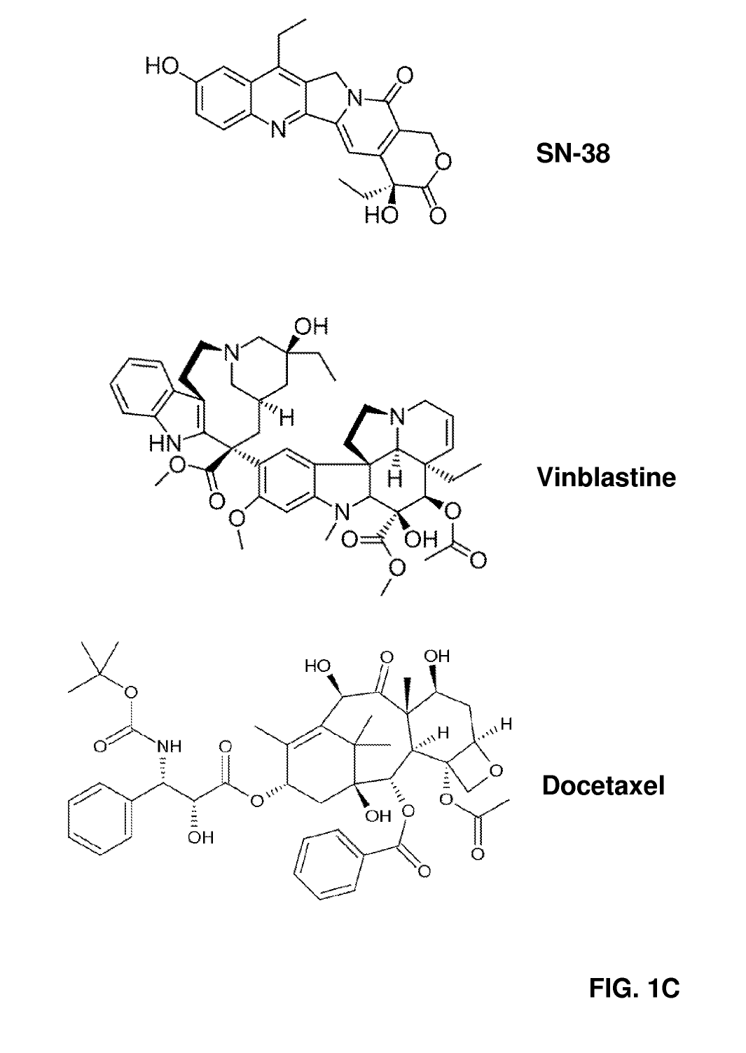

In more specific embodiments, the active agents of the present disclosure may be at least one of Cis-platin, SN-38, Vinblastine, Daunorubicin, Docetaxel, Paclitaxel, Iadarubicin, Oxaliplatin, 1,2,3,4-tetrahydronaphthalene-2,3-diamine, 2,2-dichloro-octahydrocyclohexa 1,3-diaza-2-platinacyclopentane, 2,2-dichloro-hexahydro-naphtho-1,3-diaza-2-platinacyclopentane, 4,4-dichloro-3,5-diaza-4 platinatetracycloheptadecahexaene, nitrogen mustards, spermine mustards, estrogen mustards, cholesterol mustards, combinations thereof, and derivatives thereof. The structures of some of such compounds are disclosed in FIGS. 1A-1C.

Furthermore, the active agents of the present disclosure may have various properties. For instance, in some embodiments, the active agents may be hydrophobic. See, e.g., FIGS. 1A-C. In fact, an advantage of the present invention is the effective delivery of hydrophobic active agents that may have been otherwise insoluble. As set forth in more detail below, such hydrophobic agents can be associated with various nanovectors for direct delivery to a desired tumor site without requiring the use of moieties that increase solubility but limit active agent efficacy.

The active agents of the present disclosure may also be associated with nanovectors in various manners. For instance, in some embodiments, the active agents may be non-covalently associated with nanovectors, such as through sequestration, adsorption, ionic bonding, dipole-dipole interactions, hydrogen bonding, Van der Waals interactions, and other types of non-covalent associations.

In some embodiments, the active agents may be non-covalently sequestered within a cavity, domain or surface of a nanovector. In some embodiments, the active agents may be sequestered from their surrounding environment by non-covalent association with a nanovector's solubilizing groups. In more specific embodiments where the nanovector includes hydrophobic domains and hydrophilic domains, the active agent may be associated with a hydrophobic domain. In further embodiments, a hydrophobic active agent may be associated with a hydrophobic domain of a nanovector. In some embodiments, this duality of hydrophilic and hydrophobic domains can result in the formation of structures resembling micelles or liposomes that can further entrap the active agents for delivery.

In further embodiments, the active agents of the present disclosure may be covalently associated with nanovectors. For instance, in some embodiments, the active agents of the present disclosure may be covalently associated with an active agent through a linker molecule, through a chemical moiety, or through a direct chemical bond between the active agent and the nanovector. In some embodiments, the active agent may be covalently associated with the nanovector through a cleavable moiety, such as an ester bond or amide bond. In some embodiments, the cleavable moiety may be a photo-cleavable moiety or a pH sensitive cleavable moiety. Additional modes by which active agents may be covalently or non-covalently associated with nanovectors can also be envisioned.

In some embodiments, the therapeutic compositions of the present disclosure may include a single active agent. In some embodiments, therapeutic compositions of the present disclosure may include multiple active agents.

Tracers

The therapeutic compositions of the present disclosure can also be associated with one or more tracers, such as an MRI tracer. In more specific embodiments, the tracer(s) associated with therapeutic compositions may include a gadolinium tracer, such as Gd3⁺. In further embodiments, the tracer may include, without limitation, at least one of fluorescent molecules, Qdots, radioisotopes, and combinations thereof. In various embodiments, such tracers can be used to track in real-time the location, distribution and delivery of administered therapeutic compositions. Thus, such embodiments would allow a physician to follow the degree of therapeutic composition binding to tumors, monitor the biological half-life of the therapeutic compositions, and monitor accumulation in non-target organs such the kidney and liver.

Targeting Agents

Targeting agents of the present disclosure generally refer to compounds that target a particular marker, such as markers associated with brain tumor cells. In various embodiments, the targeting agents may include, without limitation, antibodies, RNA, DNA, aptamers, small molecules, dendrimers, proteins, and combinations thereof. In more specific embodiments, the targeting agent can be a monoclonal antibody or a polyclonal antibody. In particular embodiments, the antibody may be a chimeric antibody or an antibody fragment (e.g., Fab fragment of a monoclonal antibody). In more specific embodiments, the targeting agent is an antibody directed against a marker of the brain tumor cells.

In further embodiments, the targeting agent may be an antibody that specifically targets epidermal growth factor receptors (e.g., Cetuximab). As set forth in more detail below, epidermal growth factor receptors (EGFRs) are over-expressed in many types of brain cancer cell lines. Thus, anti-EGFR antibodies and other EGFR inhibitors may be used to deliver anti-cancer agents to brain cancer cell lines in some embodiments.

Targeting agents may be associated with nanovectors in various manners. In some embodiments, targeting agents may be non-covalently associated with nanovectors, such as through sequestration, adsorption, ionic bonding, dipole-dipole interactions, hydrogen bonding, Van der Waals interactions, and other types of non-covalent associations.

In more specific embodiments, targeting agents may be non-covalently sequestered on a surface of a nanovector. In some embodiments, targeting agents may be covalently associated with nanovectors. In some embodiments, targeting agents may be covalently and non-covalently associated with nanovectors.

In more specific embodiments, the targeting agents of the present disclosure may be covalently associated with nanovectors through a linker molecule, through a chemical moiety, or through a direct chemical bond between the targeting agent and the nanovector. In some embodiments, the targeting agent may be covalently associated with the nanovector through a cleavable moiety, such as an ester bond or amide bond. In some embodiments, the cleavable moiety may be a photo-cleavable moiety or a pH sensitive cleavable moiety. Additional modes by which targeting agents may be covalently or non-covalently associated with nanovectors can also be envisioned.

Markers

As set forth previously, targeting agents of the present disclosure can target various markers associated with brain tumor cells. In some embodiments, such markers may be on a surface of brain tumor cells. In some embodiments, such markers may be within brain tumors cells. In some embodiments, such markers can include epitopes associated with brain tumor cells. In some embodiments, such epitopes may be over-expressed or up-regulated in brain tumor cells relative to other cell types.

In some embodiments, the marker is a receptor on a surface of the brain tumor cells. Examples of such receptors include, without limitation, epidermal growth factor receptors, cytokine receptors, interleukin receptors (e.g., interleukin-13), and combinations thereof. In more specific embodiments, the marker is glial fibrillary acidic protein (GFAP), a protein over-expressed in glioma cells. In further embodiments, the marker is interleukin-13 receptor (IL-13R), a cytokine receptor that is up-regulated in a large range of brain tumors, including glioblastoma multiformes (GBMs). In more specific embodiments, the marker is the epidermal growth factor receptor (EGFR), a receptor over-expressed, in either full length or truncated form, in many cancers, including GBMs. Additional markers can also be envisioned as suitable targets for brain tumors.

Brain Tumors

The therapeutic compositions of the present disclosure can be used to treat various brain tumors. In various embodiments, such brain tumors may be malignant, benign, primary, or metastatic. In some embodiments, the brain tumors to be treated may be located in different parts of the brain. In some embodiments, the brain tumors to be treated may have spread to different parts of the body.

Non-limiting examples of brain tumor types that can be treated by the methods of the present disclosure include, without limitation, gliomas, meningiomas, pituitary adenomas, and combinations thereof. Non-limiting examples gliomas include ependymomas, astrocytomas, oligodendrogliomas, mixed gliomas (e.g., oligoastrocytomas), and combinations thereof. In more specific embodiments, the therapeutic compositions of the present disclosure may be used to treat gliomas, glioblastomas, astrocytomas, neuroblastomas, retinoblastomas, meduloblastomas, oligodendrogliomas, ependymomas, choroid plexus papillomas, and combinations thereof. In more specific embodiments, the brain tumor to be treated is a primary glioblastoma multiforme (GBM).

Methods of Treating Brain Tumors

Further embodiments of the present disclosure pertain to methods of treating brain tumors in a subject. Such methods generally include administering one or more of the above-described therapeutic compositions to the subject.

Subjects

The therapeutic compositions of the present disclosure may be administered to various subjects. In some embodiments, the subject is a human being. In some embodiments, the subject is a human being with a brain tumor, such as a glioma. In some embodiments, the subjects may be non-human animals, such as mice, rats, other rodents, or larger mammals, such as dogs, monkeys, pigs, cattle and horses.

Modes of Administration

The therapeutic compositions of the present disclosure can also be administered to subjects by various methods. For instance, the therapeutic compositions of the present disclosure can be administered by oral administration (including gavage), inhalation, subcutaneous administration (sub-q), intravenous administration (I.V.), intraperitoneal administration (I.P.), intramuscular administration (I.M.), intrathecal injection, and combinations of such modes. In further embodiments of the present disclosure, the therapeutic compositions of the present disclosure can be administered by topical application (e.g, transderm, ointments, creams, salves, eye drops, and the like). Additional modes of administration can also be envisioned.

Variations

In various embodiments, the therapeutic compositions of the present disclosure may be co-administered with other therapies. For instance, in some embodiments, the therapeutic compositions of the present disclosure may be co-administered along with other anti-cancer drugs. In some embodiments, the therapeutic compositions of the present disclosure may be administered to patients undergoing chemotherapy. Other modes of co-administration can also be envisioned.

Methods of Formulating Therapeutic Compositions

Additional embodiments of the present disclosure generally pertain to methods of making therapeutic compositions of the present disclosure. Such methods generally comprise: (1) associating a nanovector with one or more active agents; and (2) associating one or more targeting agents with the nanovector. In some embodiments, one or more of the above-mentioned associations may occur non-covalently, such as by sequestration, adsorption, ionic bonding, dipole-dipole interactions, hydrogen bonding, Van der Waals interactions, and other types of non-covalent interactions. In further embodiments, one or more of the associations may occur by covalent bonding.

In various embodiments, the aforementioned associations may occur simultaneously or sequentially. In some embodiments, the associations may occur by mixing a nanovector with one or more active agents and targeting agents.

Therapeutic compositions of the present disclosure can also be formulated in conventional manners. In some embodiments, the formulation may also utilize one or more physiologically acceptable carriers or excipients. The pharmaceutical compositions can also include formulation materials for modifying, maintaining, or preserving various conditions, including pH, osmolarity, viscosity, clarity, color, isotonicity, odor, sterility, stability, rate of dissolution or release, and/or adsorption or penetration of the composition. Suitable formulation materials include, but are not limited to: amino acids (e.g., glycine); antimicrobials; antioxidants (e.g., ascorbic acid); buffers (e.g., Tris-HCl); bulking agents (e.g., mannitol and glycine); chelating agents (e.g., EDTA); complexing agents (e.g., hydroxypropyl-beta-cyclodextrin); and the like. Additional methods of formulating therapeutic compositions can also be envisioned.

Personalized Methods of Formulating Therapeutic Compositions

Additional embodiments of the present disclosure pertain to personalized methods of formulating therapeutic compositions. Such methods generally include one or more of the following steps: (1) isolating brain tumor cells from a subject; (2) determining the susceptibility of the brain tumor cells to one or more active agents; (3) determining expression levels of one or more markers of the brain tumor cells; and (4) formulating therapeutic compositions based on one or more of the aforementioned steps.

For instance, the therapeutic composition may include a nanovector and one or more active agents associated with the nanovector that were selected based on the determined susceptibility of the brain tumor cells to the active agents. Likewise, the therapeutic composition may include one or more targeting agents associated with the nanovector that have recognition activities for one or more markers of brain tumor cells that were selected based on the determined expression levels of the markers. Advantageously, such tailored methods allow for the formulation of therapeutic compositions that can specifically target tumor cells with a specified epitopic landscape for active agent delivery.

The aforementioned tailored methods of formulating therapeutic compositions have numerous variations. For instance, in some embodiments, the methods may only include a step of determining expression levels of one or more markers of the brain tumor cells and formulating therapeutic compositions based on such determinations. Likewise, in other embodiments, the methods may include only a step of determining susceptibility of the brain tumor cells to one or more active agents and formulating therapeutic compositions based on such determinations. In other embodiments, the methods may include steps of determining expression levels of one or more markers of the brain tumor cells, determining susceptibility of the brain tumor cells to one or more active agents, and formulating therapeutic compositions based on such determinations.

Likewise, various methods may be used to isolate brain tumor cells from a subject. In some embodiments, the isolation methods may include an excision of a portion of a brain tumor from the subject. In some embodiments, standard biopsy techniques may be utilized to make such excisions.

Various methods may also be used to determine the susceptibility of brain tumor cells to one or more active agents. For instance, in some embodiments, the susceptibility is determined by growing different batches of the brain tumor cells in the presence of different active agents and comparing the growth rates of the different batches with the growth rate of untreated brain tumor cells. Standard tissue culture techniques may be used for such methods. In some embodiments, one or more of the active agents that confer the slowest growth rate on tumor cells may be selected for incorporation into therapeutic compositions.

Various methods may also be used to determine the expression levels of one or more markers of the brain tumor cells. For instance, in some embodiments, the expression levels of one or more markers may be determined by treating the brain tumor cells with targeting agents that are specific for the markers. In various embodiments, standard epitope mapping techniques may be utilized for determining such expression levels. In some embodiments, the markers may be epitopes, receptors, or proteins that are over-expressed or up-regulated on the surface of brain tumor cells relative to other cells (e.g., IL-13R, GFAP, EGFR, etc.). In some embodiments, targeting agents that are selected for incorporation into therapeutic compositions may be specific for such over-expressed markers.

The personalized methods of formulating therapeutic compositions in the present disclosure may be tailored towards various subjects. In some embodiments, the subject is a human being. In some embodiments, the human being may be suffering from a brain cancer, such as glioblastoma. In further embodiments, the subject may be a non-human animal, as discussed previously.

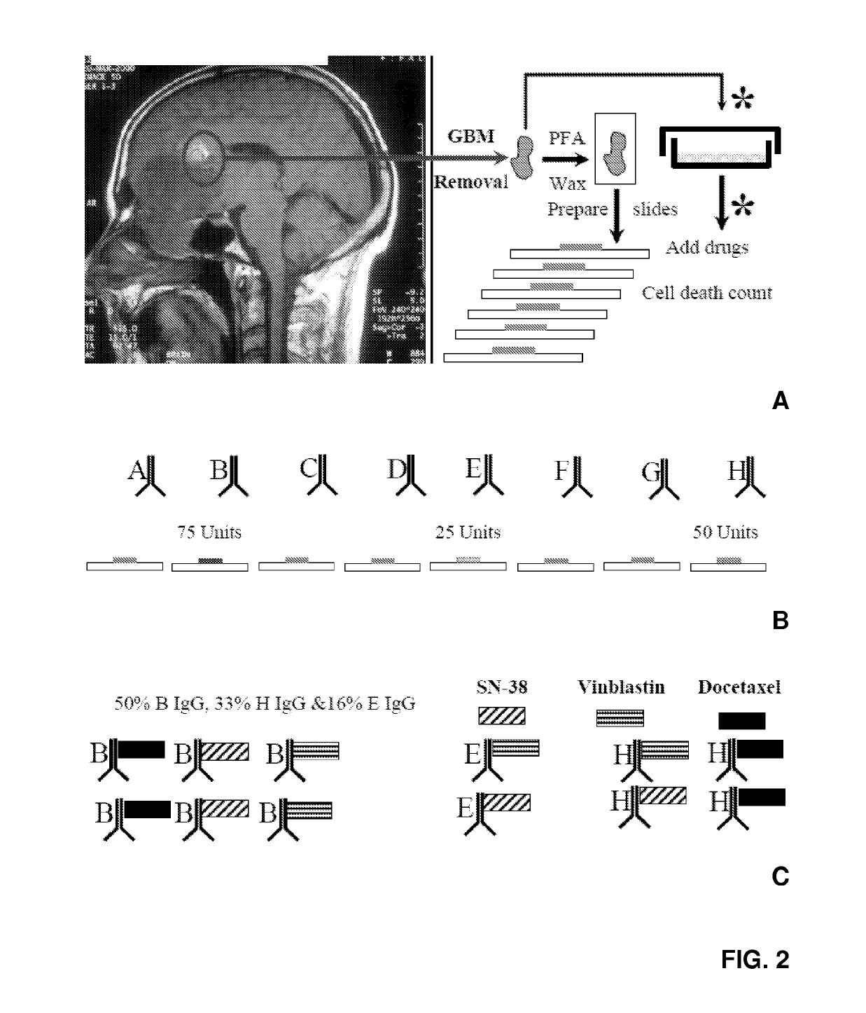

A more specific personalized method of formulating a therapeutic composition is illustrated in FIG. 2. The scheme in FIG. 2 outlines a method of formulating a therapeutic composition to treat a patient with a brain tumor (e.g., GBM). As illustrated in FIG. 2A, the brain tumor is excised by standard biopsy procedures. After excision, part of the tumor is fixed, waxed, sliced, mounted, dewaxed, and rehydrated. Part of the excised tumor can also be grown in tissue culture in order to identify the chemotherapeutic drugs to which the individual tumor is most susceptible.

As illustrated in FIG. 2B, the treated tumor slices undergo antibody screening to identify the levels of tumor-specific surface antigens in the individual tumor. Thereafter, the information obtained can be used to formulate specific therapeutic agents.

As shown in FIG. 2C, targeting agents of choice (e.g., humanized antibodies) are mixed with nanovectors (e.g., PEG-HCCs) that have been pre-loaded with active agents. Using this methodology, a large number of different active agent-loaded nanovectors can be manufactured and stored. A physician can then make an informed choice as to which active agents and targeting agents to use for a particular subject based on the attributes of the subject's tumor (e.g., expression levels of different markers and the susceptibility of tumors to various active agents).

Applications and Advantages

The therapeutic compositions and methods of the present disclosure provide numerous applications and advantages. For instance, the methods of the present disclosure can provide a facile method of manufacturing therapeutic compositions by simply mixing individual components. Furthermore, the therapeutic compositions of the present disclosure provide a method for targeted delivery of highly toxic active agents to desired sites of a tumor. Moreover, the therapeutic compositions of the present disclosure can effectively kill a majority of tumor cells without affecting normal cells. Furthermore, the therapeutic compositions of the present disclosure can be formulated according to specific attributes of a patient's brain tumor (e.g., active agent sensitivity and epitope profile). Finally, since prepared simply by mixing, the formulations can be prepared rapidly for facile patient treatment.

The methods and compositions of the present disclosure could also be used to treat various types of brain cancers. In a specific embodiment, the methods and compositions of the present disclosure could be used to treated glioblastoma. A patient diagnosed with stage 4 glioblastoma in the brain has about 9 months to live. With an intense regime of surgical removal of the accessible tumor, chemotherapy and radiation treatment, the typical time to live is still limited to about 18 months. Hence, a need remains for treating these aggressive tumors.

### Additional Embodiments

Reference will now be made to more specific embodiments of the present disclosure and experimental results that provide support for such embodiments. However, Applicants note that the disclosure below is for illustrative purposes and is not intended to limit the scope of the claimed subject matter in any way.

The Examples below pertain to hydrophilic carbon clusters (HCCs) antibody drug enhancement system (HADES), a methodology for cell-specific active agent delivery. Antigen-targeted, active agent-delivering nanovectors are manufactured by combining specific antibodies with active agent-loaded poly(ethylene glycol)-functionalized HCCs (PEG-HCCs). It is shown that HADES is highly modular as both the active agent and targeting agent component can be varied for selective killing of a range of cultured human primary glioblastoma multiforme. Using three different active agents and three different targeting agents, without covalent bonding to the nanovector, a lethality toward glioma is demonstrated with minimal toxicity toward human astrocytes and neurons.

Glioblastoma multiforme (GBM) is the most common and aggressive malignant primary brain tumors in humans. GBM prognosis is poor, with a 14 month median survival time despite interventions. Some nanovectors, such as HCCs and SWNTs, can be engineered to possess both hydrophobic and hydrophilic domains, combining high aqueous solubility with the ability to adsorb hydrophobic compounds. Therefore, nanovectors are an exciting avenue for active agent delivery of such compounds without the need for covalent active agent or covalent targeting agent attachment and could be used to target glioma and other types of brain tumors. Various forms of HCCs may be heavily oxidized carbon nanoparticles that are 30 to 40 nm long and approximately 1-2 nm wide, and although water soluble, they can be further functionalized with poly(ethylene glycol) (PEG-5000) to maintain their solubility in phosphate buffered saline (PBS), thereby rendering the PEG-HCCs nanovector system. The synthesis and characterization of PEG-HCCs has been described previously. See, e.g., PCT/US2010/054321.

PEG-HCCs have three properties that allow them to be used as nanovectors: (1) low biological toxicity with clearance mainly through the kidneys; (2) hydrophobic domains that can be non-covalently loaded with active agents; (3) and an ability to strongly bind to targeting agents (e.g., IgG-type antibodies) while the targeting agents maintain the majority of their activity. Thus, active agent-loaded PEG-HCCs combined with an IgG will bind to a chosen cell surface antigen and deliver a hydrophobic, lipophilic active agent into cells that express the selected epitope. Applicants use the nomenclature: Epitope_{AB}/Active Agent/PEG-HCCs to describe a particular hydrophilic carbon cluster antibody enhancement system (HADES) composed of a targeting agent (e.g., antibody), an active agent, and the PEG-HCCs delivery platform. In this nomenclature, non-covalent sequestration is indicated with a slash, “/”, and covalent bonding with a dash, “-”. In each case, the active agent and the targeting agent are added sequentially to the PEG-HCCs by simple mixing, thereby providing a facile “mix-and-treat” method.

In the Examples below, three potent hydrophobic active agents have been sequestered onto the PEG-HCCs. The agents were chosen on the basis of theoretical synergistic effect. These include: (a) SN-38, a topoisomerase I inhibitor, which arrests the cell cycle in the S and G2 phases; (b) Vinblastine (Vin), which causes microtubule detachment from spindle poles, arresting the cell cycle in the M phase at the mitotic spindle checkpoint; and (c) Docetaxel (Doc), which binds tubulin, preventing microtubule depolymerization and arresting the cell cycle in both the G2 and M phases, resulting in mitotic catastrophe. SN-38 can be dramatically more potent than the pro-active agent form, Irinotecan®, but the direct administration of SN-38 to patients may be problematic due to its extremely low aqueous solubility. Thus, the use of the HADES system allows for the direct delivery of this active agent, and perhaps other pharmaceutics, whose solubility requires the use of moieties that increase solubility, but limits active agent efficacy.

### Example 1

### Surface Epitope Mapping of Glioma Cell Cultures

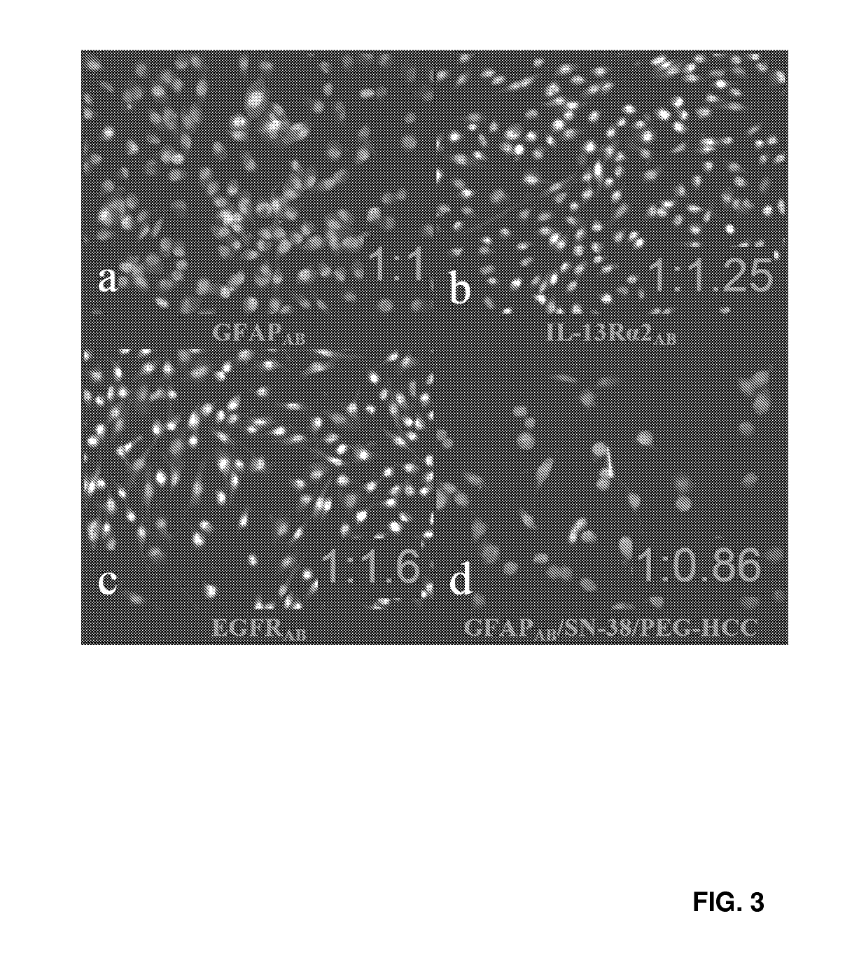

To treat GBM, immunoglobulin G antibodies (IgGs) to cell surface epitopes that are over-expressed in glioma cells relative to other cell types were selected. GFAP_{AB} is an IgG-type antibody to the glial fibrillary acidic protein (GFAP), a protein present in reactive astrocytes and also highly expressed in the majority of GBM cells. The interleukin-13 receptor (IL-13R) is a cytokine receptor, binding interleukin-13, and has been found to be up-regulated in a large range of cancers, including GBM. Normal, unreactive astrocytes express low levels of GFAP, and even lower levels of IL-13R. The epidermal growth factor receptor (EGFR) is the cell-surface receptor for members of the EGF family of extracellular proteins. This receptor is over-expressed, in either full length or truncated form, in many cancers, including GBMs. Surface epitope mapping was performed on primary glioma cell cultures. The binding of specific IgGs to GFAP:IL-13R:EGFR had ratios of 1.0:1.3:1.6, respectively. See FIGS. 3A-C.

### Example 2

### Effectiveness of IgG/Active Agent/PEG-HCCs in Killing Glioma Cells

Applicants examined the effectiveness of the antibody-targeted, IgG/Active Agent/PEG-HCCs in primary human glioma cultures and control cultures of normal human astrocytes (NHA) and human cortical neurons (HCN). As GBM generates blood-brain barrier defects, this antibody-guided active agent delivery system can be used intravenously to actively target glioma cells.

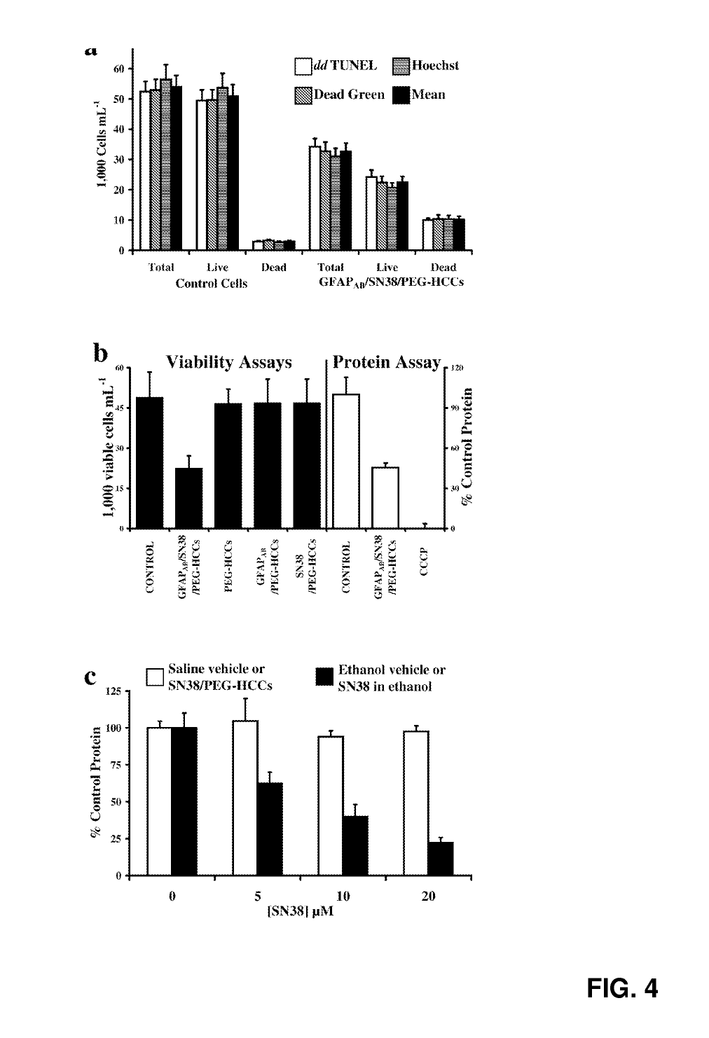

In FIG. 4A, Applicants demonstrate the ability of the HADES formulation GFAP_{AB}/SN-38/PEG-HCCs, with each component concentration at 3.9 nM, 2 μM, and 2.6 nM, respectively, to induce cell death in primary GBM cell cultures. Due to the fact that nanomaterials can often interfere with biological assays, three different methodologies were used to measure cell viability. Total, viable, and dead glioma cell numbers in confluent primary GBM cell cultures were measured using ddTUNEL (a quantitative assay for 3′ OH DNA ends), Dead Green, and Hoechst stains. Cells were treated with GFAP_{AB}/SN-38/PEG-HCCs or saline for 24 h. SN-38 induced cell death could be monitored by all three viability methodologies, but there was slight under reporting of total cell numbers using both ddTUNEL and Dead Green with respect to Hoechst, due to the presence of overlapping cells. The three methodologies are robust, even in the presence of nM concentrations of PEG-HCCs.

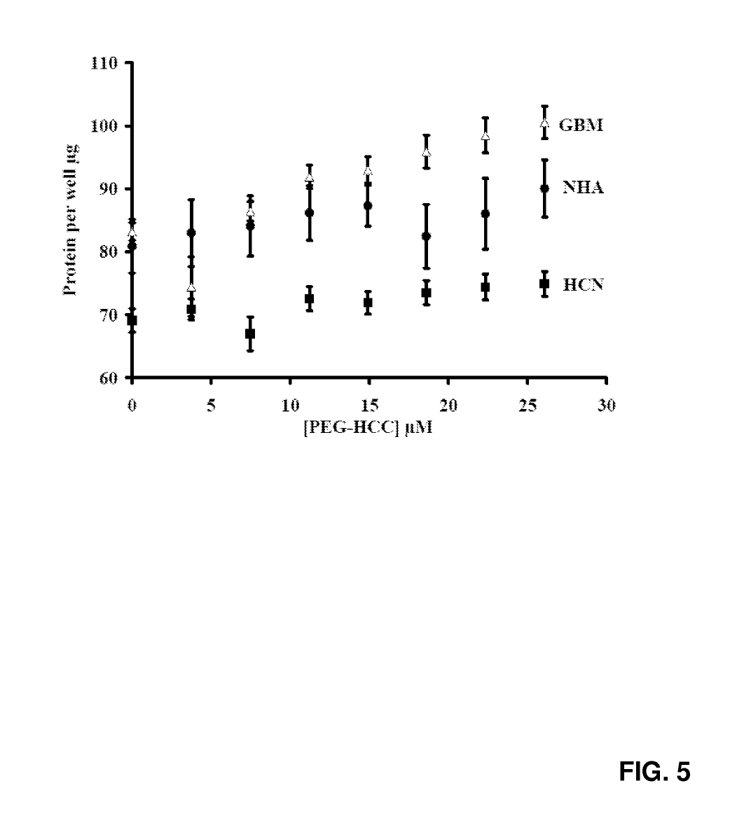

FIG. 4A further shows that in the saline control viable cell numbers increased from the ≈30,000 inoculum to 52,000 cells mL⁻¹ in 24 h, whereas incubation with GFAP_{AB}/SN-38/PEG-HCCs, there was a fall in cell numbers to only 22,000 cells mL⁻¹. Moreover, there was a three-fold increase in the number of dead cells following treatment with HADES. FIG. 4B (left panel) shows that the individual components of HADES treatment, PEG-HCCs (2.6 nM), GFAP_{AB} (3.9 nM) and SN-38 (2 μM), are not toxic towards cells when added individually. However, when the targeting antibody, active agent and nanovector are all combined, there is an increase in cell death. Addition of the three individual HADES components to glioblastoma cells results in no statistically significant difference in cell viability. Remarkably, Applicants find that PEG-HCCs alone are not toxic towards glioma, astrocytes or neurons at concentrations more than three orders of magnitude greater than those used in all the experiments related to FIG. 4. See FIG. 5.

In order to validate a high data throughput assay, Applicants compared the changes in cell numbers obtained from viability studies with the use of the bicinchoninic acid (BCA) assay of protein levels. See FIG. 4B. The maximum and minimum cellular protein levels were established using a saline negative control (100%) and carbonyl cyanide chlorophenyl hydrazone (CCCP) positive control (0%). Incubation of GBM for 24 h with CCCP (100 μM) induces cell death by mitochondrial uncoupling and allows the background matrix protein levels to be determined. Cellular protein levels following HADES treatment fell to 46% of the saline control level, mirroring the 44% levels of living cells determined using viability methodologies.

The impact of sequestering SN-38 on the hydrophobic core of the PEG-HCCs was evaluated by comparing the changes in cellular protein of GBM following 24 h incubation with SN-38/PEG-HCCs or SN-38 alone. See FIG. 4C. As mentioned previously, SN-38 is insoluble in water. For this reason, in experiments using bulk phase active agent, Applicants added either 5 μL of ethanol or ethanol containing SN-38 to each 250 μL well volume. The two controls, ethanol and saline, had no significant change in cellular protein relative to one another. Applicants found that aqueous SN-38 has an LD₅₀ of approximately 8 μM toward primary GBM, which is within the 5-10 μM range reported by others using immortalized human glioblastoma cell cultures. Interestingly, no toxicity was observed when SN-38 was presented to the cells in the form of SN-38/PEG-HCCs, even at concentrations as high as 20 μM. This indicates that the SN-38/PEG-HCCs, without antibody targeting, cannot deliver the active agent to the GBM cells at any significant rate.

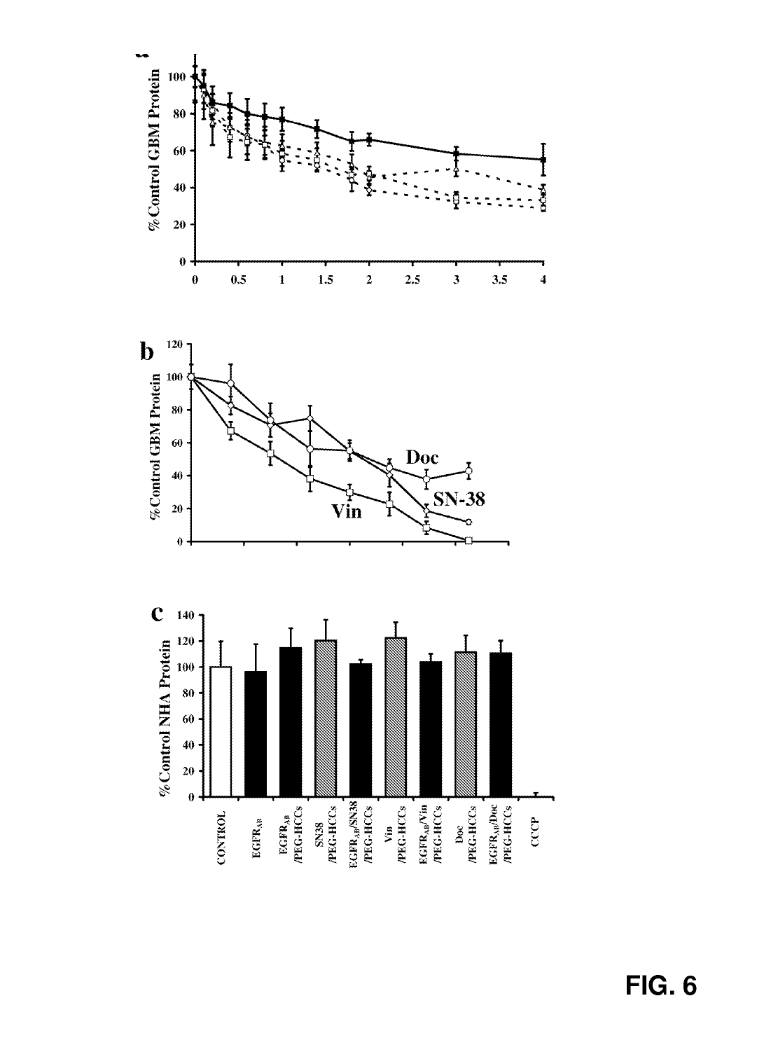

In FIG. 6, Applicants show that the HADES treatment is toxic towards a variety of human glial cell carcinomas, and that the system is flexible with respect to the loaded chemotherapeutic. In FIG. 6A, Applicants show the titration of three different primary GBM cultures, and one primary anaplastic astrocytoma (solid line) with GFAP_{AB}/SN-38/PEG-HCCs. The three GBM cultures, which have a doubling time of 28 to 34 h, have a common dose response with an LD₅₀ of 1.5 μM to 2 μM SN-38, delivered in the form of HADES. In the slower growing anaplastic astrocytoma, which has a doubling time of 48 to 52 h, the LD₅₀ is elevated to ˜3.75 μM SN-38. In FIG. 6B, Applicants show the dose response of GBM towards three different chemotherapeutics, SN-38, Vin, and Doc, which were loaded into PEG-HCCs and guided to the cell membrane using EGFR_{AB}. The highest concentration of GFAP_{AB} used on the confluent cells was 10 nM. In control experiments, Applicants incubated for 1 h with GFAP_{AB}/SN-38/PEG-HCCs, with each component concentration at 10 nM, 5 μM, and 6.5 nM, respectively. Then, fixed cells were stained using a labeled goat anti-mouse secondary antibody. Results indicated 86% saturation of the total surface GFAP epitopes, indicating that only 14% of the surface epitope is not bound to GFAP_{AB}/SN-38/PEG-HCCs. See FIG. 3D. Using this nanovector delivery system, the LD₅₀ for both SN-38 and Vin is ˜1.5 μM while for Doc it is ˜3 μM.

In FIG. 6C, Applicants show the effects of 5 μM Active Agent/PEG-HCCs ±EGFR_{AB} treatment on normal human astrocyte total protein levels, a treatment that caused >85% cell death in glioma. Neither PEG-HCCs nor EGFR_{AB}/PEG-HCCs cause cell death. Remarkably, astrocytic mass was unaffected by the three EGFR_{AB}/Active Agent/PEG-HCCs combinations, each of which was lethal to GBMs.

### Example 3

### HADES Combined Therapy

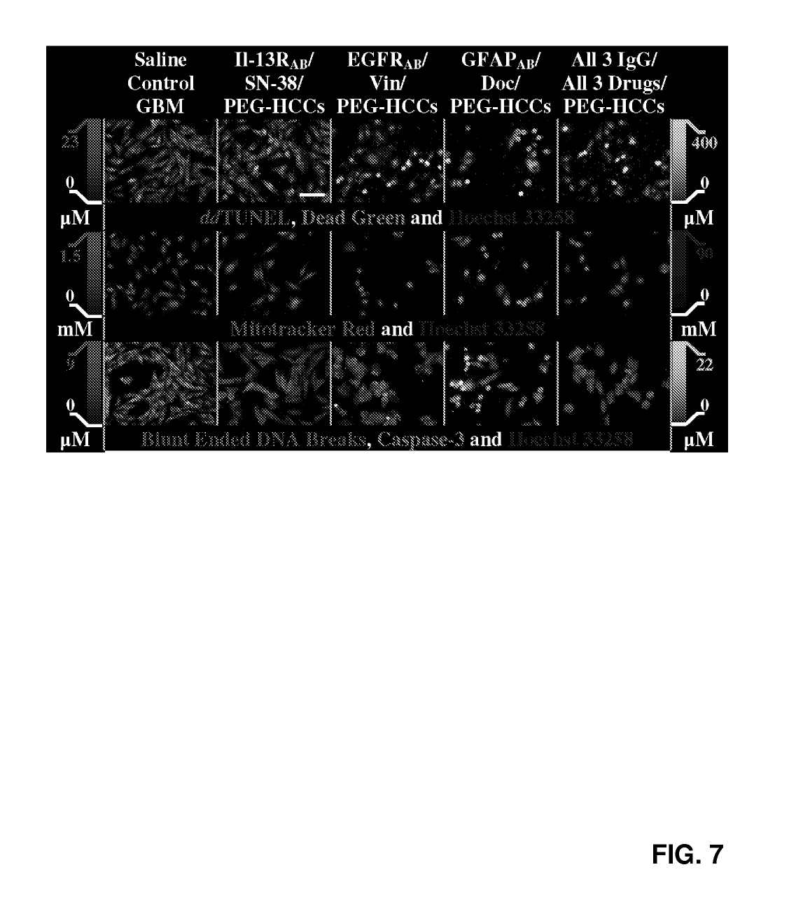

Clinically, the use of combined therapy in cancer treatment is an attempt to evade the heterogeneous response that a cancer cell population has toward different chemotherapeutics, and the ability of cancer cells to rapidly acquire active agent resistance. As SN-38, Vin, and Doc all have different pharmacologic targets, Applicants postulated that the three active agents might be able to potentiate each other's anti-cancer properties. Applicants incubated GBM, and also control NHA and HCN, with low levels of the three active agents in HADES form: consisting of three individual HADES formulations and an additional triple combination therapy where the three HADES individuals were combined. See FIG. 7. The low active agent levels chosen, 0.5 μM, allowed enough damaged and dying cells to remain at the end of a 24 h incubation to be characterized using specific probes of DNA damage, mitochondria dysfunction, loss of plasma membrane potential, and initiation of apoptotic and proteolytic cascades.

The upper panel of FIG. 7 shows the effects of the individual active agents and triple therapy on the viability of glioma primary cultured GBM cells, demonstrated by ddTUNEL (red) and Dead Green and Hoechst (blue). It is evident that both Vin and Doc have significant impacts on GBM. Microscopic examination shows evidence of mitotic catastrophe and of the presence of gear-wheel-shaped nuclei, typical of the microtubule disrupting actions of Vin and Doc. The center panel of FIG. 7 shows the loss of mitochondrial membrane potential with all four HADES regimes. Vin has been shown to alter the distribution of mitochondria throughout cells and to cause mitochondrial ‘clumping’, which is evident in GBM. Applicants also observed changes in mitochondrial morphology and cytosolic distribution in GBM treated with EGFR/Doc/PEG-HCCs that were similar to those observed in prostate cancer cells treated with Taxels.

The lowest panels of FIG. 7 show the levels of blunt ended DNA breaks and Caspase-3 activity. All three individual HADES therapies cause increases in these lethal DNA breaks and in apoptotic, Caspase-3 activity. EGFR_{AB}/Doc/PEG-HCCs in particular increase Caspase-3 activation, especially in the condensed cells, in which gear-wheel shaped nucleus predominate.

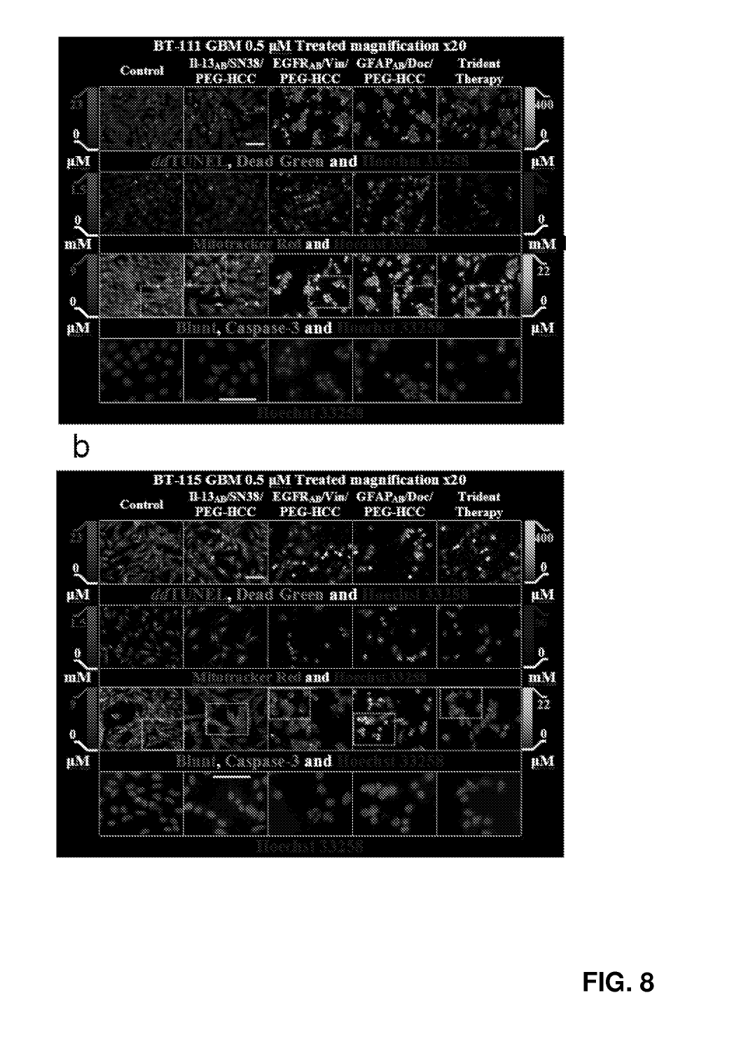

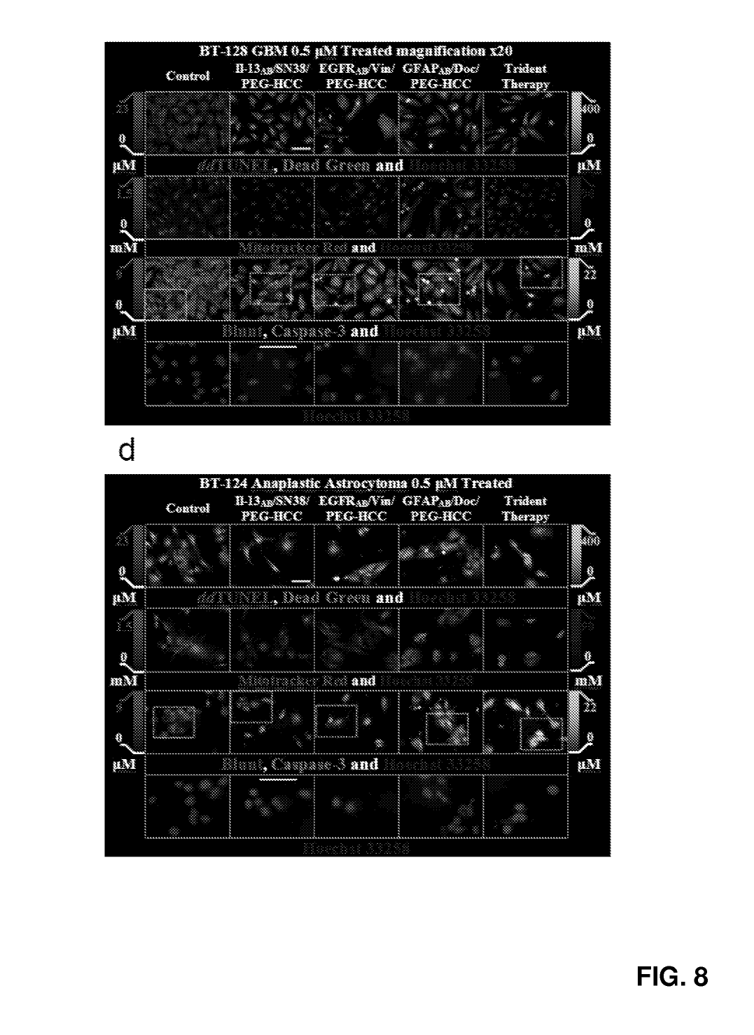

In FIGS. 8A-B, Applicants show the death labeling of two more primary GBMs and that of an anaplastic astrocytomoa, under conditions identical to that of FIG. 7. In FIGS. 8C-D, Applicants show the effects of the same therapies on cultures of normal human astrocytes (NHAs) and HCNs. In contrast to the effects of HADES on the GBMs, the effects of HADES on astrocytes and neurons are less significant. When compared to control samples, the four treatment groups demonstrate a doubling in the levels of ddTUNEL positive DNA 3′OH ends in NHA without any significant increase in cell death. It is also noteworthy that Applicants observed no changes in nuclear structure of the treated neurons, even though neurons are vulnerable towards microtubule disruption active agents like Doc and Vin.

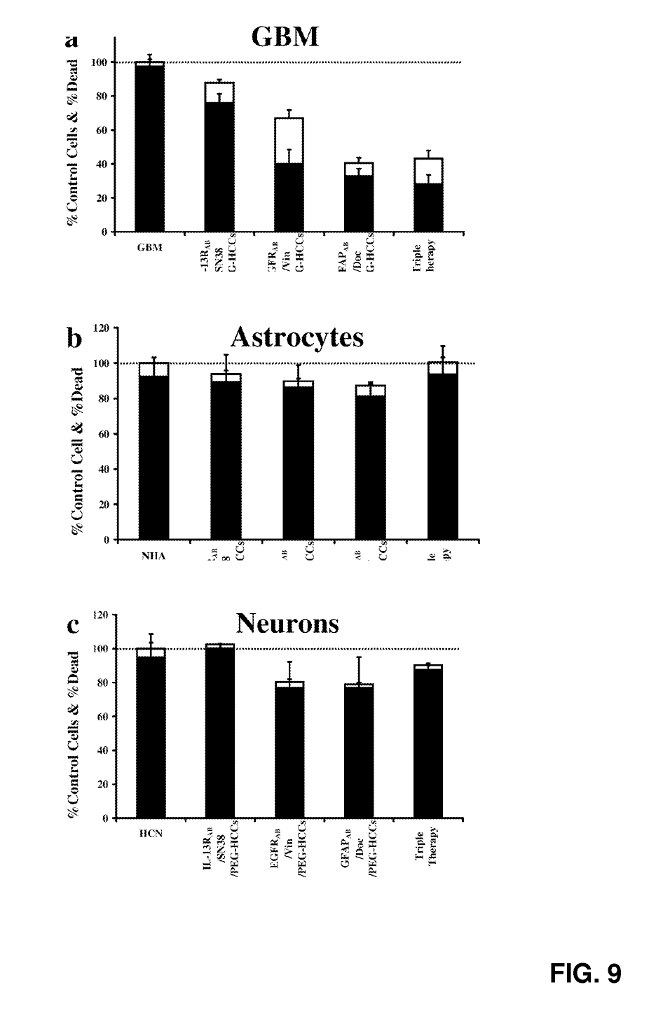

FIG. 9 shows the extent of cell viability and death for GBM, NHA and HCN using Hoechst staining. FIG. 9A shows the levels of live and dead GBM cells following individual HADES treatments and the triple therapy. Treatment with IL-13R_{AB}/SN-38/PEG-HCCs, GFAP_{AB}/Vin/PEG-HCCs or EGFR/Doc/PEG-HCCs all produced a statistically significant (p<0.01) drop in living cell numbers and an increase in dead cell percentages. There is a statistically significant (p<0.01) synergistic effect caused by triple therapy with respect to the individuals on the level of cell death.

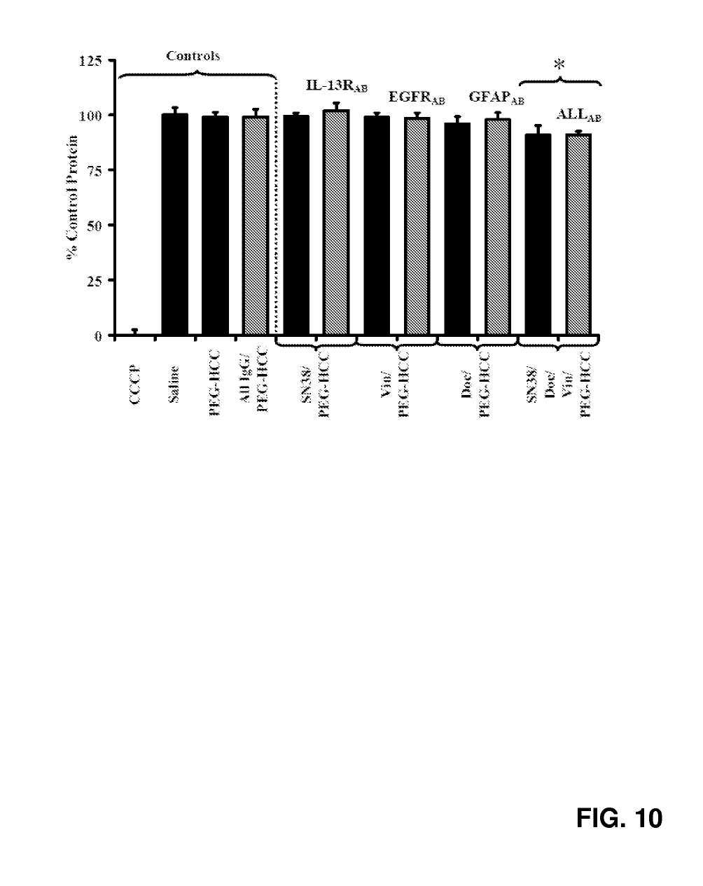

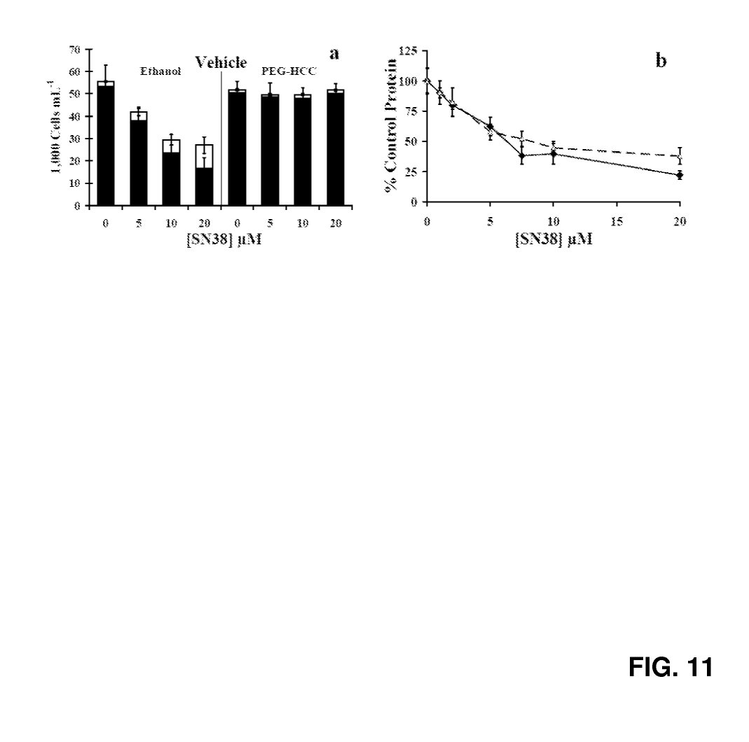

With respect to NHA and HCN, HADES treatment did not result in statistically significant changes in cell viability. However, in the case of HCN, only four wells were used for each treatment. Therefore, the number is too low to make accurate statistical assertions. Applicants therefore measured the changes in HCN protein levels in controls and following HADES treatment (as done with NHA in FIG. 6C). That data is presented in FIG. 10. The BCA assay shows that individual HADES therapies do not kill neurons, to any statistically significant degree, when using protein as a measure of cellular mass. However, combining the three active agent-loaded PEG-HCCs, in the absence or presence of antibodies, does cause a statistically significant (p<0.05, 5%) drop in cell protein levels. In spite of this increase in the killing of neurons, use of a multipronged therapy often has utility in treatment due to its potential ability to avoid the development of active agent resistance.

In summary, Applicants were able to target active agent-loaded PEG-HCCs to the surface epitopes of cells, using specific antibodies. EGFR, IL-13R and GFAP are not present in human cortical neurons, but are found in high levels in GBM. Single or triple therapy is capable of killing gliomas with extreme lethality, while at the same time causing little or no ill-effects towards either astrocytes or neurons. The simplicity of the preparation where the PEG-HCCs, active agent, and antibody are simply mixed together, coupled with the lethality of these combinations toward extremely aggressive cancers, provides encouragement for the continued testing of HADES.

### Example 4

### Materials and Methods for Examples 1-3

HCCs Functionalization, Active Agent Loading and Antibody Binding

The HCCs, PEG-HCCs and Active agent/PEG-HCCs were prepared as reported by Berlin et al. (ACS Nano 2011, 8, 6643-6650). Active agents were dissolved in a minimal amount of methanol (for Vin and Doc) or THF (for SN-38) and added dropwise into a stirring aqueous solution of PEG-HCCs. After overnight sonication, the organic solvent was removed by rotary evaporating one-third of the original volume of solution, adding one-third volume of water, and carrying out the same protocol evaporation/addition of water two more times according to published protocols (ACS Nano 2010, 4, 4621-4636). Vin (Log P 4.8) was incorporated into PEG-HCCs with a mass ratio of 5:1. Doc (Log P 2.92) was incorporated into PEG-HCCs with a mass ratio of 1.7:1. SN-38 (Log P 1.87) was incorporated into PEG-HCCs with a mass ratio of 0.33:1.

Three mouse monoclonal antibodies (IgGs) with affinities to cancer cell surface epitopes GFAP (2A5), Il-13R(YY-23Z) and EGFR (528), were obtained from Santa Cruz Biotechnology (Santa Cruz, Calif., USA). Prior to use, active agent-loaded PEG-HCCs were vortexed for 15 min and then co-incubated with the IgGs for 15 min before being diluted and added to cell media. Applicants used a mass ratio of PEG-HCCs:IgG of 4.1:1 throughout. Although heterogeneous, the average molecular mass of PEG-HCCs is ˜920,000, which gives rise to a molar PEG-HCCs:IgG ratio of 1:1.5. Assuming the binding distribution to be Poissonian, ˜80% of the PEG-HCCs have one or more IgGs bound. Visualization of mouse IgG was performed by incubating Alexa Fluor 594 goat anti-mouse IgG (Molecular Probes) overnight. The levels of Alexa Fluor-IgG were calibrated using a 5 μm thick, gelatin tissue phantom, entrapping 150 μg mL⁻¹/1 μM goat-IgG.

Cell Cultures

Primary human glioblastoma or astrocytoma cells were prepared from tumors within 10 min of their excision. The tumors were broken up using a pipette and then grown in DMEM, 20% FBS, GlutaMax-I, sodium pyruvate and Pen/Strep, for 2 weeks. After this time, and in all presented data, the same media was used, except that sodium pyruvate was omitted. NHA were obtained from Lonza (Walkersville, Md., USA) and HCN from the American Type Culture Collection (ATCC Manassas, Va. USA), and grown subject to their recommendations. NHA were grown in Astrocyte Cell Basal Medium supplemented with 3% FBS, Glutamine, Insulin, fhEGF, GA-1000 and Ascorbic acid. HCN using ATCC-formulated Dulbecco's Modified Eagle's Medium (Cat#30-2002) and supplemented with 10% FBS. GBM and NHA were grown to confluency in the appropriate media on Costar 96-well growth plates (Corning, N.Y.C, NY, USA). HCN were grown on 16-well Lab-Tek slide chambers (Nalge Nunc, Rochester, N.Y., USA). Cells were grown for 24 h in the presence or absence of all effectors, in a total volume of 250 μL.

Assays

The ability of PEG-HCCs to take up hydrophobic solutes compromises a large number of high throughput proliferation assays. Applicants find that many common reporter chromophore/fluorophores partition into PEG-HCCs and then undergo altered absorbance/fluorescence properties. PEG-HCCs also interfere with peptide-bond chelated copper reduction of Folin-Ciocalteu reagent (phosphomolybdate/phosphotungstate).

Protein Measurement

Cell proliferation studies with PEG-HCCs included four HCN controls: 100 μM CCCP (100% cell death), saline vehicle, PEG-HCCs and IgG/PEG-HCCs using monoclonal antibodies toward GFAP, IL-13R or EGFR. Three HADES treatments where PEG-HCCs loaded with the active agents Vin, Doc, or SN-38 were added to HCN with or without antibodies. The HADES treatment also included a triple therapy with or without antibodies. After 24 h, the cells were washed with PBS, solubilized using 0.1% SDS and then the protein present in the well was measured using the Thermo Scientifics Micro Bicinchoninic acid (BCA) Assay Kit (Waltham, Mass., USA). The data is displayed in FIG. 8.

Cell Viability Measurements

The measurements and quantification of DNA 3′OH and blunt ended breaks by use of the ddTUNEL and blunt ended ligation were performed as described in our recent publications. The biotinylated ddUTP and biotinylated blunt ended oligonucleotide probe was visualized using Texas Red labeled avidin. Cells were incubated with 500 nM Mitotracker Red (Cat#M22425), 1 μM Hoechst 33258 (Cat#H1398) and 100 nM Dead Green (Cat#I10291), with reagents obtained from Molecular Probes (Eugene, Oreg., USA). The activity of Caspase-3 in fixed, 0.1% Triton permeabilized cells was measured using the Molecular Probes R110-EnzChek Assay Kit (Cat#E13184), incubating cells for 1 h at 37° C. Signals from Dead Green/R110 and from Mitotracker were calibrated against known concentrations of liquid FITC-gelatin and Texas Red-gelatin and then against FITC/Texas Red gelatin tissue phantoms 5 μm in thickness.

Viability Cut-Off

Cells were counted at 4× magnification using a Nikon Eclipse TE2000-E fluorescent microscope equipped with a CoolSnap ES digital camera system (Roper Scientific) containing an CCD-1300-Y/HS 1392×1040 imaging array cooled by a Peltier device. Images were recorded using Nikon NIS-Elements software as JEP2000 files. Cells were deemed to be non-viable if they had Dead Green/Hoechst signals >5 times the level found in control cells and >4.2 times the level of ddTUNEL labeled DNA 3′OH ends in control cells.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A therapeutic composition for treating a brain tumor, wherein the therapeutic composition comprises:
a nanovector;
an active agent associated with the nanovector, wherein the active agent has activity against brain tumor cells; and
a targeting agent associated with the nanovector, wherein the targeting agent has recognition activity for a marker of the brain tumor cells.

2. The therapeutic composition of claim 1, wherein the active agent is non-covalently associated with the nanovector.

3. The therapeutic composition of claim 1, wherein the active agent is covalently associated with the nanovector.

4. The therapeutic composition of claim 1, wherein the targeting agent is non-covalently associated with the nanovector.

5. The therapeutic composition of claim 1, wherein the targeting agent is covalently associated with the nanovector.

6. The therapeutic composition of claim 1, wherein the nanovector comprises hydrophobic domains and hydrophilic domains, and wherein the active agent is associated with the hydrophobic domains.

7. The therapeutic composition of claim 1, wherein the nanovector is selected from the group consisting of single-walled nanotubes, double-walled nanotubes, triple-walled nanotubes, multi-walled nanotubes, ultra-short nanotubes, graphene, graphene nanoribbons, graphite, graphite oxide nanoribbons, carbon black, oxidized carbon black, hydrophilic carbon clusters and combinations thereof.

8. The therapeutic composition of claim 1, wherein the nanovector is functionalized with a plurality of solubilizing groups.

9. The therapeutic composition of claim 8, wherein the solubilizing groups are selected from the group consisting of polyethylene glycols, polypropylene glycols, poly(p-phenylene oxide), polyethylene imines, poly(vinyl alcohol), poly(acrylic acid), poly(vinyl amines) and combinations thereof.

10. The therapeutic composition of claim 1, wherein the nanovector is an ultra-short single-walled nanotube, and wherein the nanotube is functionalized with a plurality of solubilizing groups.

11. The therapeutic composition of claim 1, wherein the nanovector is a polyethylene glycol functionalized hydrophilic carbon cluster (PEG-HCC).

12. The therapeutic composition of claim 1, wherein the active agent is selected from the group consisting of small molecules, proteins, DNA, antisense oligonucleotides, miRNA, siRNA, aptamers, and combinations thereof.

13. The therapeutic composition of claim 1, wherein the active agent is hydrophobic.

14. The therapeutic composition of claim 1, wherein the active agent is selected from the group consisting of Cis-platin, SN-38, Vinblastine, Daunorubicin, Paclitaxel, Docetaxel, Iadarubicin, Oxaliplatin, 1,2,3,4-tetrahydronaphthalene-2,3-diamine, 2,2-dichloro-octahydrocyclohexa 1,3-diaza-2-platinacyclopentane, 2,2-dichloro-hexahydro-naphtho-1,3-diaza-2-platinacyclopentane, 4,4-dichloro-3,5-diaza-4-platinatetracycloheptadecahexaene, nitrogen mustards, spermine mustards, estrogen mustards, cholesterol mustards, and combinations thereof.

15. The therapeutic composition of claim 1, wherein the marker of the brain tumor cells comprises an epitope on a surface of the brain tumor cells.

16. The therapeutic composition of claim 1, wherein the marker of the brain tumor cells is glial fibrillary acidic protein (GFAP).

17. The therapeutic composition of claim 1, wherein the marker of the brain tumor cells is a receptor on a surface of the brain tumor cells, wherein the receptor is selected from the group consisting of epidermal growth factor receptors, cytokine receptors, interleukin receptors, and combinations thereof.

18. The therapeutic composition of claim 1, wherein the targeting agent is selected from the group consisting of antibodies, proteins, RNA, DNA, aptamers, small molecules, dendrimers, and combinations thereof.

19. The therapeutic composition of claim 1, wherein the targeting agent is an antibody directed against a marker of the brain tumor cells.

20. The therapeutic composition of claim 1, wherein the brain tumor to be treated is selected from the group consisting of gliomas, glioblastomas, astrocytomas, neuroblastomas, retinoblastomas, meduloblastomas, oligodendrogliomas, ependymomas, choroid plexus papillomas, meningiomas, pituitary adenomas, and combinations thereof.

21. The therapeutic composition of claim 1, wherein the brain tumor to be treated is a primary glioblastoma multiforme (GBM).

22. A method of treating a brain tumor in a subject, wherein the method comprises:
administering a therapeutic composition to the subject, wherein the therapeutic composition comprises:
a nanovector;
an active agent associated with the nanovector, wherein the active agent has activity against brain tumor cells, and
a targeting agent associated with the nanovector, wherein the targeting agent has recognition activity for a marker of the brain tumor cells.

23. The method of claim 22, wherein the subject is a human being.

24. The method of claim 22, wherein the administering of the therapeutic composition comprises intravenous administration.

25. The method of claim 22, wherein the nanovector is selected from the group consisting of single-walled nanotubes, double-walled nanotubes, triple-walled nanotubes, multi-walled nanotubes, ultra-short nanotubes, graphene, graphene nanoribbons, graphite, graphite oxide nanoribbons, carbon black, oxidized carbon black, hydrophilic carbon clusters and combinations thereof.

26. The method of claim 22, wherein the nanovector is an ultra-short single-walled nanotube, wherein the nanotube is functionalized with a plurality of solubilizing groups.

27. The method of claim 22, wherein the nanovector is a polyethylene glycol functionalized hydrophilic carbon cluster (PEG-HCC).

28. The method of claim 22, wherein the active agent is selected from the group consisting of small molecules, proteins, DNA, antisense oligonucleotides, miRNA, siRNA, aptamers, and combinations thereof.

29. The method of claim 22, wherein the active agent is selected from the group consisting of Cis-platin, SN-38, Vinblastine, Daunorubicin, Docetaxel, Paclitaxel, Iadarubicin, Oxaliplatin, 1,2,3,4-tetrahydronaphthalene-2,3-diamine, 2,2-dichloro-octahydrocyclohexa 1,3-diaza-2-platinacyclopentane, 2,2-dichloro-hexahydro-naphtho-1,3-diaza-2-platinacyclopentane, 4,4-dichloro-3,5-diaza-4-platinatetracycloheptadecahexaene, nitrogen mustards, spermine mustards, estrogen mustards, cholesterol mustards, and combinations thereof.

30. The method of claim 22, wherein the marker of the brain tumor cells is a receptor on a surface of the brain tumor cells.

31. The method of claim 22, wherein the targeting agent is selected from the group consisting of antibodies, proteins, RNA, DNA, aptamers, small molecules, dendrimers, and combinations thereof.

32. The method of claim 22, wherein the targeting agent is an antibody directed against a marker of the brain tumor cells.

33. The method of claim 22, wherein the brain tumor to be treated is selected from the group consisting of gliomas, glioblastomas, astrocytomas, neuroblastomas, retinoblastomas, meduloblastomas, oligodendrogliomas, ependymomas, choroid plexus papillomas, meningiomas, pituitary adenomas, and combinations thereof.

34. The method of claim 22, wherein the brain tumor to be treated is a primary glioblastoma multiforme (GBM).

35. A method of formulating a therapeutic composition for treating a brain tumor in a subject, wherein the method comprises:
isolating brain tumor cells from the subject;
determining expression levels of one or more markers of the brain tumor cells; and
formulating the therapeutic composition, wherein the formulated therapeutic composition comprises:
a nanovector;
an active agent associated with the nanovector; and
a targeting agent associated with the nanovector, wherein the targeting agent has recognition activity for a marker of the brain tumor cells, and wherein the targeting agent is selected based on the determined expression levels of the one or more markers of the brain tumor cells.

36. The method of claim 35, further comprising a step of determining susceptibility of the brain tumor cells to one or more active agents, and selecting the active agent based on the determined susceptibility of the brain tumor cells to the one or more active agents

37. The method of claim 36, wherein the susceptibility of the brain tumor cells to one or more active agents is determined by growing different batches of the brain tumor cells in the presence of different active agents and comparing growth rates of the different batches with the growth rate of untreated brain tumor cells.

38. The method of claim 35, wherein the isolating of the brain tumor cells comprises an excision of a portion of a brain tumor from the subject.

39. The method of claim 35, wherein the expression levels of one or more markers of the brain tumor cells are determined by treating the brain tumor cells with targeting agents that are specific for the markers.

40. The method of claim 35, wherein the subject is a human.
